(19) United States
(12) Patent Application Publication
(10) Pub. No.: US 2026/0195994 A1
(43) Pub. Date: Jul. 9, 2026

(54) METHOD OF ENHANCING USER COMPLIANCE IN VR STIMULATION THERAPY FOR THE TREATMENT OF NEURODEGENERATIVE DISEASES

(71) Applicant: Clarity Technologies SASU, Paris (FR)

(72) Inventors: Carolina Reis, Paris (FR); Raphael Certain, Paris (FR)

(21) Appl. No.: 19/445,388

(22) Filed: Jan. 9, 2026

Related U.S. Application Data
(60) Provisional application No. 63/743,574, filed on Jan. 9, 2025.

(51) Int. Cl. G06T 19/00; G16H 20/70

(52) U.S. Cl. CPC G06T 19/00; G16H 20/70

(57) ABSTRACT

The present disclosure relates to systems and methods for engagement-aware delivery of neuromodulation-based therapeutic sessions using immersive computing devices. A therapeutic system delivers sensory, audiovisual, or multimodal neuromodulation protocols within immersive or semi-immersive environments and applies engagement-supporting elements, including gamification assets, to promote sustained participation and adherence across repeated sessions. During session delivery, user interaction and responsiveness are monitored using sensing components integrated with or operatively coupled to the system. Engagement-related data is used to assess execution fidelity and compliance and to apply engagement-supporting adjustments that preserve the intended neuromodulation protocol. Session-level execution and compliance information may be stored and reported to authorized parties to support continuity of therapy. The disclosed systems enable reliable, engagement-aware neuromodulation therapy delivery in home-based or unsupervised settings without requiring continuous clinical supervision.

### CROSS-REFERENCE TO RELATED APPLICATION

The present patent application claims the priority benefit of U.S. provisional patent application number 63/743,574 filed January 9, 2025, the disclosure of which is incorporated by reference herein.

### FIELD OF THE DISCLOSURE

The present disclosure relates to systems and methods for delivering neuromodulation-based therapeutic sessions using immersive computing devices, and more particularly to engagement-aware neuromodulation systems that monitor user interaction and apply engagement-supporting adjustments to promote execution fidelity, compliance, and reliable delivery of prescribed stimulation protocols

### BACKGROUND

Many therapeutic interventions for brain disorders require repeated and sustained exposure over extended periods of time in order to achieve meaningful clinical benefit.In particular, neuromodulation-based therapies—such as sensory stimulation, neurofeedback, or other other neurostimulation-based approaches—are increasingly used to treat neurological, neurodegenerative, and neuropsychiatric disorders by repeatedly influencing neural activity, circuit dynamics, or network-level function. The magnitude and durability of therapeutic effects in such interventions may vary substantially across individuals and over time and may depend on factors such as attention, engagement, and consistency of participation across repeated treatment sessions. Expected therapeutic outcomes may include improvements in cognitive function, behavioral symptoms, mood, sleep, motor control, or other functional domains, depending on the condition being treated and the nature of the intervention. In many embodiments, such therapies are intended to be administered longitudinally, often over weeks or months, to achieve durable effects and sustained benefit. Such interventions are often implemented using digital, device-based, or immersive platforms.

Such therapeutic interventions may be applied across a range of brain disorders, including neurodegenerative, cerebrovascular, demyelinating, traumatic, and neuropsychiatric conditions. The interventions may encompass neuromodulatory, sensory, cognitive, behavioral, or functional therapies, alone or in combination, and may involve structured tasks, instructions, or interactive elements. In many embodiments, these therapies are delivered using digital, wearable, or immersive platforms, such as virtual reality (VR), augmented reality (AR), or mixed-reality systems, to support therapeutic delivery and, in some cases, monitoring.

Increasingly, these therapeutic interventions are delivered outside of traditional clinical environments, including in home or other unsupervised or semi-supervised settings. While such delivery models may improve accessibility and scalability, they introduce challenges related to adherence, engagement, and verification of therapy execution. Clinical teams may have limited visibility into how consistently, attentively, or completely a patient engages with a prescribed therapy between supervised visits, making it difficult to determine whether suboptimal outcomes or non-response arise from insufficient therapeutic efficacy, inadequate exposure, or reduced compliance with the treatment plan.

A significant limitation of existing therapy delivery systems is their reliance on user compliance and sustained engagement to achieve an effective therapeutic dose. Patients with brain disorders may experience fatigue, apathy, attentional deficits, reduced motivation, cognitive overload, or emotional dysregulation, which can impair their ability or willingness to complete prescribed therapeutic tasks. As a result, therapy sessions may be prematurely terminated, partially completed, or performed with insufficient attention or effort, even when the underlying therapeutic modality is otherwise appropriate. These failures of compliance and engagement may substantially reduce therapeutic effectiveness and complicate interpretation of clinical outcomes.

In therapeutic systems that incorporate structured tasks, instructions, or interactive components, user compliance directly affects whether the intended therapeutic intervention is delivered as prescribed. Incomplete task execution, reduced attention, or failure to follow instructions may result in partial, inconsistent, or insufficient delivery of the intended therapeutic dose, particularly in interventions that require repeated or temporally structured exposure over time. This includes neuromodulation-based therapies in which therapeutic efficacy may depend on consistent delivery of stimulation parameters, timing, or task-linked engagement across sessions. In addition, when such systems are configured to assess behavioral performance, physiological signals, or other outcome measures, inadequate compliance may compromise the validity, reliability, or interpretability of collected data. As a result, insufficient monitoring of compliance and execution fidelity may obscure whether observed clinical outcomes—whether symptom-based or biomarker-based—reflect true lack of therapeutic efficacy or inadequate delivery of the prescribed intervention.

In neuromodulation-based therapies, engagement and attention may further influence therapeutic impact beyond simple session completion. Neuromodulation approaches—including sensory stimulation, neurofeedback, or other stimulation paradigms—may interact with neural activity in ways that are sensitive to user attention, arousal state, or task engagement. As used herein, sensory stimulation refers to the delivery of controlled sensory inputs—such as visual, auditory, tactile, or multisensory stimuli—configured to influence neural activity, network dynamics, or functional state through patterned, rhythmic, or temporally structured presentation. Sensory stimulation may be delivered alone or in combination with interactive content, tasks, or environments, and may be configured to support neuromodulation objectives associated with treatment of brain disorders. For example, attention to sensory stimuli has been associated with modulation of neural entrainment magnitude, which may contribute to downstream cognitive, behavioral, or motor effects in certain therapeutic paradigms. Similarly, pairing neuromodulation with interactive or task-based elements may engage reward, attentional, or motivational systems that influence how stimulation is processed at the network level. While such effects may vary across individuals and conditions, they underscore engagement as a controllable variable that may affect both delivery fidelity and therapeutic efficacy in neuromodulation-based interventions.

In this context, the systems and methods described herein are directed to the delivery of therapeutic interventions and are not limited to monitoring-only applications. In various embodiments, the system delivers therapeutic content, stimulation, or task-based interventions intended to produce clinically meaningful outcomes, while also incorporating monitoring functions to assess symptoms, behavioral responses, or physiological signals. Such monitoring may support personalization, adaptation, and evaluation of therapy and may benefit from high levels of user compliance and engagement.

Accordingly, there is a need for therapeutic systems that explicitly address compliance and engagement as core technical challenges in the delivery of treatment for brain disorders. Such systems may monitor adherence and engagement during therapy sessions and may modify or adapt therapeutic content, task structure, or gamification strategies during a session, between sessions, or across multiple therapeutic sessions over time. In some embodiments, the system may further facilitate communication with clinicians, caregivers, or other authorized parties to support supervision, feedback, or intervention across sessions. By addressing compliance as a system-level function, therapeutic platforms may improve the reliability of monitoring data, enhance effective delivery of therapeutic dose, and optimize clinically meaningful outcomes across a broad range of brain disorders.

### SUMMARY OF THE DISCLOSURE

The present disclosure relates to systems and methods for engagement-aware delivery of neuromodulation-based therapeutic interventions for therapeutic intervention in brain disorders, including, by way of example and not limitation, neurodegenerative, neurological, neuropsychiatric, or neurodevelopmental conditions. The disclosed embodiments are directed to improving execution fidelity, adherence, and compliance of therapeutic delivery during repeated intervention sessions, particularly in home-based or unsupervised environments. In various embodiments, a therapeutic system delivers neuromodulation-based interventions through an interactive device-based platform, such as a virtual, augmented, or mixed-reality environment. Therapeutic interventions may comprise sensory, audiovisual, or multimodal neurostimulation paradigms embedded within immersive or semi-immersive environments and may be structured using task logic or interaction rules to engage, modulate, or intended to target specific behaviors, cognitive functions, or brain networks. Engagement-supporting elements, including gamification strategies, may be layered on top of the therapeutic protocol to promote motivation, sustained participation, and adherence over repeated sessions. During delivery of therapeutic sessions, multimodal input data indicative of user interaction, attention, arousal, or responsiveness may be captured via sensing components integrated with or operatively coupled to the delivery system.

Such data may be processed to generate reports indicative of session execution, engagement, execution fidelity, or compliance, and such reports may be stored, presented, or shared with one or more authorized parties, including the patient, caregivers, or clinical teams, to support monitoring, interpretation, and continuity of therapy delivery. Through these capabilities, the disclosed embodiments enable reliable, engagement-aware delivery of neuromodulation-based therapy by supporting sustained participation, execution fidelity, and continuity of prescribed therapeutic regimens across repeated sessions without requiring continuous direct clinical supervision.

### BRIEF DESCRIPTIONS OF THE DRAWINGS

FIG. 1:Illustrates a system architecture for delivering, monitoring, adapting and reporting engagement-aware neuromodulation therapeutic sessions using an immersive or semi-immersive device, according to an embodiment.

FIG. 2: Illustrates an example operational flow for configuring and delivering an engaging neuromodulation session with layered engagement-supporting elements, according to an embodiment.

FIG. 3: Illustrates an example operational flow of an engagement module configured to monitor, score, and support execution fidelity and compliance during and across neuromodulation sessions, according to an embodiment.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples. As used herein, an “engaging neuromodulation session” refers to a structured intervention session in which one or more neuromodulation or sensory stimulation protocols are delivered to a user via a headset-based system, together with engagement-supporting elements including interactive environments, task logic, or gamification mechanisms, such that engagement, attention, and execution fidelity are actively supported during delivery of the neuromodulation protocol.

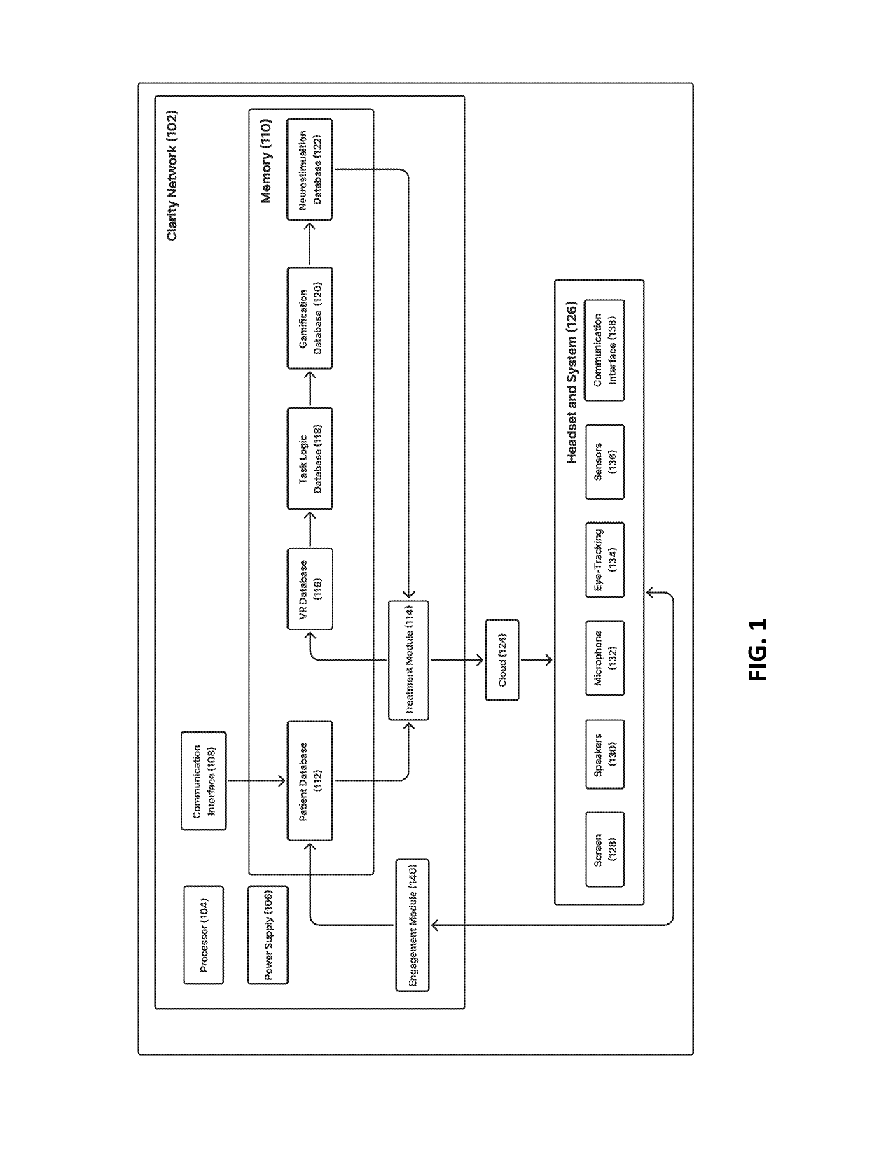

FIG. 1 illustrates a method for enhancing user compliance in VR stimulation therapyfor use in neuromodulation-based therapies. . This method comprises a clarity network 102 that enables the integration of patient performance analysis, scoring, and gamification processes with databases for cognitive tasks, neurostimulation protocols, virtual reality environments, and gamification methods to deliver engaging configured neurostimulation sessions. In some embodiments, stimulation protocols are selected prior to session execution. During session execution, one or more presentation parameters of the stimulation may be adjusted to support delivery fidelity, user attention, comfort, or safety, without adapting the stimulation based on therapeutic response or symptom-related outcome. The clarity network 102 contains a response module 110, recording module 112, and scoring module 114, which manage the delivery of VR environments and cognitive tasks, collect and analyze patient data, and integrate gamification elements with neurostimulation protocols based on performance metrics. The clarity network 102 includes a virtual reality database 118 that stores immersive VR scenarios, which may include emotionally relevant content tailored to the patient a neurostimulation database 120 that provides predefined stimulation protocols tailored to therapeutic goals, a cognitive task database 122 that contains a library of cognitive, motor, and functional tasks, a gamification database 126 that stores gamification methods for enhancing patient engagement, and a patient database 124 that maintains patient-specific data, scores, and session histories. The clarity network 102 connects with the headset 130, delivering data in real-time to enable precise, engaging, and reliable and consistent therapy delivery..

Further, embodiments may include a processor 104, also known as a central processing unit (CPU), which may facilitate the operation of the system according to the instructions stored in the memory 116. The processor 104 may include suitable logic, circuitry, interfaces, and/or code that may be configured to execute a set of instructions stored in the memory 116. The processor 104 may be a hardware component that performs arithmetic, logic, and control operations on data. The processor 104 may be comprised of the arithmetic logic unit, control unit, memory subsystems, and other subsystems. The processor 104 may be responsible for performing arithmetic and logical operations on data. The processor 104 may include components for addition, subtraction, multiplication, and division and logical operations such as AND, OR, and NOT. The processor 104 may be responsible for fetching instructions from memory 116, decoding them, and executing them. The processor 104 may manage the flow of data between different components of the system as a whole, ensuring that operations are performed in the correct order and that data is transferred efficiently. The processor 104 may provide fast access to frequently used data and instructions. The processor 104 may include components such as caches, registers, and pipelines, which are designed to minimize the time required to access and manipulate data. The processor 104 may include various other components and subsystems, such as instruction set architecture (ISA), which may define the set of instructions that the processor 104 can execute. The processor 104 may specify the format of instructions and data, the addressing modes used to access memory 116 and I/O devices, and the interrupt and exception handling mechanisms used to manage errors and other events. The processor 104 may include advanced instruction execution capabilities, support for virtualization and parallel processing, and power management mechanisms that reduce energy consumption and heat dissipation.

Further, embodiments may include a communication interface 106, which may be a hardware or software component that enables communication between two or more electronic devices or systems. The communication interface 106 may include a set of protocols, rules, and standards that define how information is transmitted and received between the devices. The communication interface 106 may be a physical connector, wireless network, or software application and may include components such as drivers, software libraries, and firmware that may be used to control and manage the communication process. In some embodiments, the communication interface 106 may be compatible with USB, Bluetooth, or Wi-Fi. The communication interface 106 may communicate with a network. Examples of networks may include, but are not limited to, the Internet, a cloud network, a Wireless Fidelity (Wi-Fi) network, a Wireless Local Area Network (WLAN), a Local Area Network (LAN), a telephone line (POTS), Long Term Evolution (LTE), and/or a Metropolitan Area Network (MAN). Further, embodiments may include a communication interface 106, which may be a hardware or software component that enables communication between two or more electronic devices or systems. The communication interface 106 may include a set of protocols, rules, and standards that define how information is transmitted and received between devices. The communication interface 106 may be a physical connector, wireless network, or software application and may include components such as drivers, software libraries, and firmware that are used to control and manage the communication process. In some embodiments, the communication interface 106 may be compatible with USB, Bluetooth, or Wi-Fi. The communication interface 106 may communicate with one or more networks, including the Internet, a cloud network, a Wireless Fidelity (Wi-Fi) network, a Wireless Local Area Network (WLAN), a Local Area Network (LAN), a telephone line (POTS), Long Term Evolution (LTE), or a Metropolitan Area Network (MAN). In some embodiments, the communication interface 106 may support transmission of information generated by the system to one or more external systems or authorized users for purposes of monitoring, review, oversight, documentation, or record-keeping. Such information may include, by way of example and not limitation, session execution status, session completion indicators, engagement or compliance metrics, timestamps, alerts, or summary information derived from operation of the system. In some embodiments, information transmitted via the communication interface 106 may be provided during or after a therapy session and may be received by caregivers, clinicians, administrators, or other authorized parties through one or more external devices or platforms. The transmitted information may be used for reporting, assessment, or supervision purposes outside the scope of therapy delivery. In some embodiments, the communication interface 106 may support periodic or event-based notifications indicative of session execution conditions or compliance-related events. Such notifications are generated for informational purposes and do not modify, interrupt, or otherwise affect delivery of therapeutic content, stimulation protocols, or task execution during an ongoing session.

Further, embodiments may include a power supply 108, which may be an electrical device or system that is used to convert electrical power from a source to a specific form or voltage that can be utilized by an electronic or electrical device. The power supply 108 may be designed to regulate and control the output power to ensure that the device or system receives the correct amount of power without any damage. The power supply 108 may include a variety of components, such as transformers, rectifiers, filters, voltage regulators, and control circuits that work together to provide the desired output voltage and current. The input power source can be from an AC or DC power source such as a battery, wall outlet, or generator. The power supply 108 may be classified based on various parameters such as the type of output voltage, power rating, efficiency, regulation, and application. In some embodiments, the power supply 108 may include various protection mechanisms such as overvoltage protection, overcurrent protection, short-circuit protection, and thermal protection to ensure safe and reliable operation.

Further, embodiments may include a memory 110 configured to store data, instructions, and persistent system information used by the Clarity Network 102 during operation. In some embodiments, the memory 110 stores data generated or received during delivery of interactive virtual-reality–based therapeutic sessions, including sensor-derived data, engagement-related measures, session metadata, and other information supporting longitudinal tracking and analysis of user participation and compliance. In some embodiments, the memory 110 stores one or more databases used by the Clarity Network 102, including a patient database 112, a virtual reality database 116, a task logic database 118, a gamification database 120, and a neurostimulation database 122, as described herein. The memory 110 may further store executable instructions that, when executed by the processor 104, cause the system to perform the coordination, scoring, recording, and data-handling operations described in this disclosure. The memory 110 may be implemented using one or more non-transitory computer-readable storage media located locally within the Clarity Network 102, remotely via cloud-based storage resources 124, or in a distributed configuration combining local and remote storage. Such storage media may include, by way of example and not limitation, random access memory (RAM), read-only memory (ROM), solid-state storage, hard disk drives, removable storage media, or combinations thereof.

Further, embodiments may include a patient database 112 configured to store patient-specific information and longitudinal records associated with operation of the Clarity Network 102. In some embodiments, the patient database 112 stores identifiers, configuration parameters, session histories, engagement measures, compliance metrics, performance-related outputs, therapeutic schedules, and metadata generated during delivery of virtual-reality–based therapeutic sessions.

In some embodiments, the patient database 112 may further store information received from external sources via the communication interface 108, including clinician-provided inputs, caregiver observations, user-reported information, or other externally sourced data associated with monitoring of therapy execution or user participation. Such information may be structured and stored for subsequent retrieval, reporting, or review by authorized system components or external users.

The patient database 112 may serve as a persistent repository through which engagement-related outputs generated by the engagement module 140 and externally sourced inputs may be maintained over time. In this manner, the patient database 112 supports longitudinal tracking of therapy execution, user compliance, and engagement across multiple sessions without requiring continuous direct supervision during session delivery.

Further, embodiments may include a treatment module 114 configured to assemble and deliver interactive therapeutic sessions through the Clarity Network 102. The treatment module 114 may retrieve predefined session components from one or more data repositories, including the virtual reality database 116, task logic database 118, gamification database 120, and neurostimulation database 122, and combine such components to form a structured therapeutic session. In some embodiments, the treatment module 114 assembles session content based on predefined session configurations, user selections, or stored scheduling information. Stimulation protocols used during a session may be selected prior to session initiation and incorporated into the assembled session content as predefined components for the session. The treatment module 114 may cause the assembled session content to be delivered to the user via the headset and system 126 through the cloud 124. During delivery of the session, the headset and system 126 may generate data indicative of user interaction, responsiveness, attention proxies, or task execution behavior through one or more sensors, including eye-tracking components, microphones, or physiological sensors. In some embodiments, limited adjustments to session delivery may be performed during session execution to support delivery fidelity, user attention, comfort, or safety. Such adjustments may include, by way of example and not limitation, modification of presentation characteristics, pacing, or sensory salience, without altering the therapeutic objective, stimulation protocol selection, or symptom-specific treatment logic of the session. Data generated during session delivery may be provided to the engagement module 140 for assessment of user engagement, task execution fidelity, or compliance. The treatment module 114 operates independently of engagement scoring and does not modify therapeutic intent or stimulation protocol selection based on engagement assessment during the session.

In some embodiments, the memory 112 may include a Virtual Reality (VR) database 116 configured to store immersive or semi-immersive environments, scenes, audiovisual content, or other presentation elements used during engaging neuromodulation sessions. The VR database 116 may include content suitable for delivery via immersive, semi-immersive or augmented platforms and may support presentation of therapeutic, assessment, or engagement-supporting experiences during intervention sessions.

In some embodiments, the VR database 116 may store multiple variants of environments or content elements that differ in visual, auditory, or interactive characteristics, including, by way of example and not limitation, complexity, pacing, sensory intensity, or emotional valence. Content stored in the VR database 116 may include fictitious or abstract environments, naturalistic or real-world scenes, externally sourced or generated multimedia content such as music, videos, movies, or games, as well as personalized media such as user-selected images, photographs, audio recordings, or video content.

Content retrieved from the VR database 116 may be selected, sequenced, or substituted by the treatment module 114 based on predefined session configurations or engagement-related outputs generated by the engagement module 140. In some embodiments, presentation characteristics of retrieved content may be adjusted to support user attention, comfort, or delivery fidelity, without modifying therapeutic intent or stimulation protocol selection during a session. Such selection or adjustment may occur during session assembly, between sessions, or as part of predefined engagement-supporting logic.

In some embodiments, the memory 112 may include a task logic database 118 configured to store logic, rules, or parameters governing interaction flow during engaging neuromodulation sessions. The task logic database 118 may define, by way of example and not limitation, sequencing of content, timing of events, handling of user interactions, session progression, or transitions between presentation elements during intervention sessions.

In some embodiments, task logic stored in the task logic database 118, in combination with content retrieved from the virtual reality database 116, may be used to create interactive experiences that impose varying levels of cognitive, motor, or functional demand on a user. Such task logic may range from minimal or implicit interaction handling—such as passive exploration or simple responsiveness to user actions—to more structured and complex tasks involving explicit objectives, multi-step interactions, or coordinated cognitive or motor activities.

In other embodiments, when structured tasks are included as part of an intervention protocol, the task logic database 118 may store definitions, parameters, or progression rules associated with such tasks. In some embodiments, task logic retrieved from the task logic database 118 may be selected and executed by the treatment module 114 in accordance with predefined session configurations. In some embodiments, one or more execution parameters of a task—such as difficulty level, pacing, repetition, cueing, or presentation timing—may be adjusted during session execution based on engagement-related outputs generated by the engagement module 140, in order to support delivery fidelity, user attention, comfort, or sustained compliance.

Such adjustments do not alter the therapeutic intent, task identity, or task objectives associated with the session, and do not introduce new therapeutic logic during a session. Rather, the adjustments are limited to presentation or execution characteristics that facilitate continued participation and completion of the prescribed task.

Across sessions, content or task variants stored in the task logic database 118 may be selected or sequenced based on predefined session structure, historical engagement patterns, or user-specific configuration, while preserving the therapeutic role of the tasks within the intervention protocol.

In some embodiments, the memory 110 may include a gamification database 120 configured to store gamification strategies, engagement-supporting elements, and presentation rules used to promote sustained user participation, adherence, and delivery fidelity during engaging neuromodulation sessions. The gamification database 120 may define how therapeutic or assessment content is presented to a user in a manner intended to support engagement across individual sessions and over extended durations, including longitudinal use over weeks or months. In some embodiments, the gamification database 120 may store one or more gamification strategies associated with a therapeutic protocol generated by the treatment module 114 or, where applicable, an assessment or testing protocol. Such strategies may be defined at multiple levels, including within individual tasks, across sets of tasks within a session, or across sessions over time, and may be selected based on predefined session structure, intended therapeutic context, or user-specific engagement history. The gamification database 120 may store, by way of example and not limitation, engagement-supporting mechanisms such as point systems, progress indicators, levels, badges, achievements, challenges, rewards, feedback cues, narrative elements, visual or auditory reinforcement, pacing mechanisms, progression cues, or combinations thereof. In some embodiments, gamification mechanisms may operate explicitly or implicitly, including embodiments in which feedback or reinforcement is delivered without requiring explicit user awareness of scoring or reward structures. In some embodiments, gamification content, parameters, or strategies retrieved from the gamification database 120 may be selected, substituted, or reconfigured based on engagement-related outputs generated by the engagement module 140. Such selection or reconfiguration may be applied during an engaging neuromodulation session, between sessions, or across multiple sessions over time, and may include adjustment of reward schedules, feedback frequency, pacing of progression, challenge framing, or presentation style. Such adjustments are configured to support continued engagement and compliance without modifying therapeutic objectives, stimulation parameters, or underlying task definitions during a session. In some embodiments, gamification strategies may influence the timing, presentation, or reinforcement of task progression defined by the task logic database 118, while preserving the therapeutic intent and structural definition of the tasks themselves. In some embodiments, engagement-related outputs, compliance indicators, or session summaries generated by the system may be transmitted via the communication interface 138 to one or more external systems or authorized parties for review. Such external parties may include clinicians, caregivers, or support personnel associated with the user. The transmitted information may include, by way of example and not limitation, session completion status, engagement scores, adherence metrics, or longitudinal summaries of interaction with the system.

In some embodiments, the memory 110 may include a neurostimulation database 122 configured to store parameters, protocols, or definitions associated with delivery of neuromodulation-based stimulation during engaging neuromodulation sessions. The neurostimulation database 122 may support one or more non-invasive neuromodulation paradigms and may provide predefined stimulation protocols accessed by the treatment module 114 to configure delivery of stimulation via the headset and system 126. In some embodiments, neuromodulation paradigms stored in the neurostimulation database 122 may comprise sensory stimulation delivered in visual, auditory, or multisensory modalities. Such sensory stimulation may include, by way of example and not limitation, rhythmic or periodic visual stimulation delivered via a display, patterned auditory stimulation delivered via speakers or headphones, or coordinated audiovisual stimulation presented synchronously or asynchronously. In some embodiments, coordinated audiovisual stimulation may be delivered with defined temporal relationships including synchronous delivery, asynchronous delivery, alternating delivery, or ordered delivery in which stimulation in one modality precedes or follows stimulation in another modality according to a specified schedule. In some embodiments, stimulation delivery may include purposeful interruptions, pauses, or segment boundaries incorporated into a stimulation protocol to support session structure, user comfort, safety constraints, or engagement-supporting presentation, while preserving the neuromodulation modality and therapeutic intent defined for the session. Stimulation parameters stored in the neurostimulation database 122 may include stimulus frequency, temporal modulation pattern, duty cycle, intensity, spatial extent, or synchronization across modalities. In some embodiments, stimulation parameters stored in the neurostimulation database 122 may define multi-frequency stimulation paradigms in which two or more stimulation frequencies are delivered concurrently, sequentially, or hierarchically. For example, a first stimulation frequency may be embedded within, amplitude-modulated by, phase-modulated by, or otherwise coordinated with a second stimulation frequency, including embodiments in which slower rhythmic stimulation is combined with faster rhythmic stimulation to define nested or composite temporal structure. In some embodiments, stimulation waveforms may comprise sinusoidal modulation, square-wave modulation, pulse trains, isochronic modulation, or combinations thereof, and may include defined phase relationships or phase offsets between modalities or between frequency components. Such multi-frequency or waveform-defined stimulation paradigms may be delivered in visual, auditory, or multisensory form and may be represented as predefined protocols selectable by the treatment module 114 in accordance with a prescribed intervention protocol. In some embodiments, the neurostimulation database 122 may store stimulation protocols configured prior to session execution and intended to be delivered repeatedly across one or more engaging neuromodulation sessions. By way of example, a stimulation protocol may comprise visual or audiovisual stimulation delivered at frequencies corresponding to a predefined neuromodulation paradigm, including flicker-based or grating-based visual stimulation patterns. Such stimulation may be embedded within immersive or interactive environments retrieved from the virtual reality database 116 and may be delivered with or without structured interaction defined by the task logic database 118. In some embodiments, stimulation protocols retrieved from the neurostimulation database 122 may be delivered in conjunction with engagement-supporting elements retrieved from the gamification database 120. Engagement-related outputs generated by the engagement module 140 may be used to support adherence, execution fidelity, and completion of prescribed intervention sessions incorporating stimulation protocols retrieved from the neurostimulation database 122. In some embodiments, the neurostimulation database 122 may additionally store definitions associated with multiple neuromodulation modalities, including visual stimulation, auditory stimulation, tactile or vibrotactile stimulation, somatosensory stimulation, or peripheral nerve stimulation, or electrical or electromagnetic stimulation, including transcranial alternating current stimulation (tACS), transcranial direct current stimulation (tDCS), transcranial random-noise stimulation (tRNS), temporal interference stimulation, transcranial magnetic stimulation (TMS), or combinations thereof. In some embodiments, one or more such neuromodulation modalities may be delivered alone or in combination with sensory stimulation paradigms and may be embedded within virtual-reality environments, tasks, or gamified content presented via the headset and system 126. Selection of a neuromodulation modality may be defined prior to session execution in accordance with a prescribed intervention protocol. Engagement-supporting mechanisms described herein are configured to promote adherence to delivery of the prescribed intervention without modifying the therapeutic intent or neuromodulation modality during a session.

In some embodiments, the system may include a headset and system 126 configured to deliver engaging neuromodulation sessions and to collect data indicative of user interaction, responsiveness, and engagement during such sessions. The headset and system 126 may serve as a primary delivery and monitoring interface of the Clarity Network 102, enabling presentation of immersive or device-based content and acquisition of engagement-related signals during intervention sessions. The headset and system 126 may include one or more output components, such as a display 128 and speakers 130, configured to present visual, auditory, or audiovisual content retrieved from the virtual reality database 116 and, in some embodiments, combined with a neuromodulation paradigm retrieved from the neurostimulation database 122. Such content may be delivered in accordance with an intervention protocol generated by the treatment module 114. In some embodiments, the display 128 may comprise a head-mounted or near-field display configured to render immersive, semi-immersive or augmented environments. The headset and system 126 may further include one or more input components configured to capture observable user interaction with the delivered content. Such components may include, by way of example and not limitation, one or more microphones 132 for detecting speech or vocal responses, eye-tracking components 134 for monitoring gaze behavior or visual attention, and additional sensors 136 such as cameras, inertial sensors, accelerometers, gyroscopes, or other input devices configured to capture movement, orientation, or interaction-related signals during an engaging neuromodulation session. Data captured by the headset and system 126 may be transmitted, in real time or near real time, to the Clarity Network 102 via one or more communication interfaces 138. Such data may be provided to the engagement module 140 for analysis and scoring of engagement, attention, task execution fidelity, or compliance, as described herein. Engagement-related outputs generated by the engagement module 140 may be transmitted back to the headset and system 126 via the communication interface 138 to control or adjust engagement-supporting presentation elements during session execution, while preserving the therapeutic intent, neuromodulation modality, and session objective defined by the treatment module 114. In some embodiments, the headset and system 126 may perform limited local processing of captured data to support low-latency interaction handling, stimulus presentation stability, or session control, while higher-level engagement assessment and adaptation logic is performed by the Clarity Network 102 or associated cloud computing resources 124. In some embodiments, the headset and system 126 may support delivery of neuromodulation-based interventions, including sensory stimulation delivered via visual or auditory output components. During session execution, one or more presentation characteristics of delivered stimulation—such as intensity, salience, timing, spatial distribution, or sensory emphasis—may be adjusted based on engagement-related signals captured by the headset and system 126, in order to support user attention, comfort, safety, and execution fidelity. Such adjustments are limited to delivery or presentation characteristics and do not modify the therapeutic intent, neuromodulation modality, or intervention objective associated with the session. The headset and system 126 may communicate with the Clarity Network 102 through wired or wireless interfaces and may support operation in connected or intermittently connected deployment scenarios. Power may be provided through rechargeable batteries or external power sources, and the headset and system 126 may include safety-related features or constraints to support extended or repeated use during longitudinal intervention regimen.

In some embodiments, the headset and system 126 may include a screen 128 configured to function as a primary visual interface for delivery of engaging neuromodulation sessions within immersive, semi-immersive, or augmented-reality environments. The screen 128 may support presentation of therapeutic or intervention-related content, delivery of visual sensory stimulation, and display of user-facing guidance or experience elements intended to support engagement, execution fidelity, and adherence. The screen 128 may comprise a high-performance near-field or head-mounted display panel, such as a liquid crystal display (LCD), organic light-emitting diode (OLED), micro-OLED, waveguide-based display, or other suitable display technology compatible with immersive or augmented-reality systems. In some embodiments, the screen 128 may support high resolution, high brightness, and high refresh rates sufficient to enable delivery of temporally structured visual stimulation, including periodic or rhythmic luminance modulation, while maintaining visual stability and minimizing perceptual artifacts. In some embodiments, the screen 128 may be configured to support temporally structured visual stimulation at one or more target frequencies by operating at a refresh rate sufficient to represent the intended temporal modulation with adequate fidelity, including embodiments in which the refresh rate is selected to be at least twice a target stimulation frequency and, in some embodiments, an integer multiple of the target stimulation frequency to reduce temporal jitter and improve stability of periodic luminance modulation. In some embodiments, the temporal modulation implemented by the screen 128 may comprise square-wave, sinusoidal, or other periodic luminance modulation profiles, and may be implemented as full-field modulation, spatially localized modulation, or object-based modulation within an immersive environment, with modulation depth and duty cycle selected according to a prescribed intervention protocol.

In some embodiments, the screen 128 may present visual stimuli delivered as full-field modulation, spatially localized modulation, object-based modulation, texture-embedded patterns, or overlays integrated within immersive or augmented environments. In some embodiments, visual stimulation presented via the screen 128 may be defined in terms of an active region in which temporally structured luminance modulation is presented and an inactive region in which temporally structured luminance modulation is reduced or absent, thereby enabling delivery of stimulation within a portion of the user’s field of view while preserving other portions of displayed content for comfort, legibility, or task execution. The active region may correspond to a full field, a spatially localized region, or one or more rendered objects, and may be positioned centrally, peripherally, or dynamically repositioned within the displayed environment. In some embodiments, placement or size of an active region may be selected or updated based on gaze-related signals captured by the eye-tracking component 134 to maintain retinal projection consistency of the active region, to ensure task-relevant content remains visible, or to support sustained attention while preserving the therapeutic intent and stimulation protocol defined for the session. Visual content may be presented as part of environments retrieved from the virtual reality database 116 and may be coordinated with auditory output or other neuromodulation modalities during an engaging neuromodulation session. The screen 128 may support adjustment of visual presentation parameters including, by way of example and not limitation, brightness, contrast, color balance, spatial extent, modulation depth, salience, or temporal characteristics of presented content. Such parameters may be configured in accordance with neuromodulation protocols retrieved by the treatment module 114 and may be adjusted during session execution to support delivery fidelity, user attention, comfort, or safety, without modifying the therapeutic intent of the intervention. In some embodiments, the screen 128 may present instructional or experience-supporting elements intended to promote adherence and execution fidelity, including task instructions, session timing cues, progress indicators, attentional prompts, confirmations of completion, or motivational feedback. Such elements may be updated dynamically in response to detected engagement or attention-related signals during or between sessions to support continued participation and compliance with prescribed session parameters. In some embodiments, visual content presented on the screen 128 may be modified during or between engaging neuromodulation sessions to maintain engagement, reduce fatigue, or support completion of prescribed session durations. Through these capabilities, the screen 128 enables reliable delivery of immersive and interactive visual content while supporting execution fidelity and sustained user participation over repeated intervention sessions.

In some embodiments, the headset and system 126 may include one or more speakers 130 configured to function as an audio interface for delivery of engaging neuromodulation sessions, including presentation of therapeutic auditory stimulation, delivery of task-related instructions or guidance, provision of engagement-supporting feedback, and rendering of immersive environmental audio content. The speakers 130 may comprise integrated audio output devices positioned near the user’s ears or implemented using bone-conduction, spatial-audio, or other audio transduction technologies suitable for immersive or augmented-reality use and extended session durations. In some embodiments, the speakers 130 may support a wide acoustic frequency range, low distortion, and sufficient temporal fidelity to preserve the timing and modulation characteristics of auditory signals used in neuromodulation-based intervention protocols. In some embodiments, the speakers 130 may deliver auditory stimulation as part of neuromodulation protocols retrieved from the neurostimulation database 122. Such auditory stimulation may include, by way of example and not limitation, rhythmic tones, amplitude-modulated sounds, pulse trains, click sequences, or other temporally structured auditory signals configured to support neuromodulatory objectives. Auditory stimulation may be delivered alone or in coordination with visual stimulation presented via the screen 128, including synchronous or asynchronous audiovisual presentation as defined by a prescribed intervention protocol. In addition to therapeutic stimulation, the speakers 130 may present instructional or experience-supporting audio elements intended to promote adherence and execution fidelity. Such elements may include spoken instructions, alerts, prompts, confirmations, reminders, progress cues, or motivational messages presented during intervention sessions to assist users in following prescribed session structure, timing, or task requirements. In some embodiments, audio content delivered via the speakers 130 may be adjusted during or between engaging neuromodulation sessions to support user attention, comfort, or compliance with prescribed session parameters. Such adjustments may include modification of timing, repetition, volume, or salience of audio cues, while maintaining consistency with the therapeutic intent and neuromodulation protocol defined for the session. The speakers 130 may further render immersive environmental audio content as part of virtual or augmented environments retrieved from the virtual reality database 116, including ambient sounds, music, spatialized cues, or contextual soundscapes that contribute to immersion and sustained user participation. Through these capabilities, the speakers 130 support reliable delivery of neuromodulation-based auditory stimulation, presentation of guidance and feedback, and enhancement of immersive intervention experiences, thereby contributing to execution fidelity, adherence, and sustained compliance across repeated intervention sessions.

In some embodiments, the headset and system 126 may include one or more microphones 132 configured to capture audio signals generated by a user during engaging neuromodulation sessions. The microphone 132 may function as an input interface through which user speech, vocal responses, or other audible interactions are detected and used to support monitoring of engagement, attention, task execution fidelity, and longitudinal compliance. The microphone 132 may capture audio data continuously or intermittently during testing or treatment sessions, including during periods in which the user is interacting explicitly with system prompts or passively receiving therapeutic or engagement-supporting content, thereby enabling background collection of interaction-related information without requiring interruption of the session. Audio signals captured by the microphone 132 may be transmitted to the Clarity Network 102 and provided to the engagement module 140 for analysis. Such analysis may occur without requiring explicit user action and without altering the therapeutic content or task objectives of the session. In some embodiments, audio data captured via the microphone 132 may be generated as a result of task-defined user interactions during an engaging neuromodulation session, such as verbal responses to prompts, spoken confirmations, acknowledgments, or other vocal interactions associated with execution of prescribed tasks. Such audio data may be analyzed by the engagement module 140 to derive indicators of engagement, attention, responsiveness, or participation. In some embodiments, engagement-related information derived from microphone data may be used in conjunction with gamification strategies retrieved from the gamification database 120 to provide engagement-supporting feedback, reinforcement cues, reminders, or progression indicators intended to support adherence and completion of prescribed session activities. In some embodiments, audio-derived engagement indicators may be evaluated in combination with other engagement-related data captured by the headset and system 126, including eye-tracking data, motion-related signals, or physiological measurements, to support generation of composite engagement or compliance measures by the engagement module 140. The microphone 132 may further support voice-based interaction mechanisms used for guidance or confirmation during intervention sessions, including spoken instructions, prompts, acknowledgments, or feedback presented to the user via the speakers 130. In some embodiments, voice-based interaction may form part of engagement-supporting presentation elements, such as requiring verbal acknowledgment to confirm attention or task completion. In some embodiments, engagement-related information derived from microphone data may be used by the engagement module 140 to support adjustment of engagement-supporting elements of session delivery, including modification of prompts, pacing, feedback timing, or presentation cues delivered via the screen 128 or speakers 130, while preserving the therapeutic intent, task identity, and intervention parameters defined for the session. Through these capabilities, the microphone 132 provides a non-intrusive channel for collection of interaction-related data and supports voice-based engagement mechanisms, thereby contributing to reliable execution, delivery fidelity, and sustained compliance across repeated engaging neuromodulation sessions.

In some embodiments, the headset and system 126 may include an eye-tracking component 134 configured to capture data indicative of user gaze behavior during engaging neuromodulation sessions. The eye-tracking component 134 may function as an input interface through which visual attention, responsiveness, and interaction with presented content are monitored to support assessment of engagement, execution fidelity, and longitudinal compliance. The eye-tracking component 134 may capture gaze-related data continuously or intermittently during intervention sessions, including during periods of active interaction or passive content delivery, and such data may be collected unobtrusively in the background while visual content, instructions, or sensory stimulation are presented via the screen 128, without requiring explicit user action. In some embodiments, gaze-related data captured by the eye-tracking component 134 may include, by way of example and not limitation, gaze direction, fixation duration, fixation stability, saccade patterns, dwell time on defined regions of interest, frequency or duration of gaze diversion away from presented content, blink rate, or prolonged eye closure, and such measures may be indicative of visual attention, responsiveness to presented stimuli, task participation, or interruption of session execution. Eye-tracking data may be transmitted to the Clarity Network 102 and provided to the engagement module 140, where such data may be analyzed alone or in combination with other engagement-related inputs, such as audio data captured by the microphone 132 or motion-related signals captured by sensors 136, to derive engagement or attention indicators reflecting whether a user is visually attending to task-relevant content or maintaining gaze patterns consistent with intended session execution. In some embodiments, engagement-related outputs derived from eye-tracking data may be used by gamification strategies retrieved from the gamification database 120 to provide feedback, reminders, motivational cues, or progression indicators intended to support sustained attention and adherence, without altering underlying task logic or therapeutic intent. In some embodiments, eye-tracking-derived engagement information may be used by the engagement module 140 to support adjustment of engagement-supporting presentation elements, including modification of content presentation, pacing, prompts, or attention cues delivered via the screen 128 or speakers 130, while preserving the therapeutic intent, intervention objectives, and neuromodulation modality defined for the session. Through these capabilities, the eye-tracking component 134 provides a non-intrusive source of information indicative of user attention and interaction, supporting reliable execution of engaging neuromodulation sessions and improved interpretability of longitudinal engagement and adherence.

In some embodiments, the headset and system 126 may include one or more sensors 136 configured to capture non-intrusive physiological, behavioral, or motion-related signals during engaging neuromodulation sessions. The sensors 136 may be integrated into the headset and system 126 or may comprise external or peripheral sensing devices operatively coupled to the system and used to assess user responsiveness, arousal state, attention, or continuity of session execution. In some embodiments, sensors 136 may include pupillometry sensors configured to capture pupil size or pupil dynamics as proxies for alertness or cognitive engagement, inertial sensors such as accelerometers or gyroscopes for detecting head or body movement, cameras for capturing facial orientation or gross behavioral posture, and physiological sensors configured to capture signals related to heart rate, heart-rate variability, respiratory rate, electrodermal conductance, body temperature, or muscle activity, using corresponding sensing modalities such as pulse oximeters, electrocardiography sensors, galvanic skin response sensors, respiratory sensors, temperature sensors, or electromyography sensors. Data captured by sensors 136 may be transmitted to the Clarity Network 102 and provided to the engagement module 140 for analysis, independently or in combination with data captured by the microphone 132 or eye-tracking component 134, to derive engagement-, attention-, or compliance-related indicators reflecting how closely a user’s observed interaction aligns with intended execution of an engaging neuromodulation session. Such indicators may be used to assess whether the user remains attentive, responsive, and engaged throughout delivery of prescribed session content and to support adjustment of engagement-supporting presentation elements during or between sessions, without modifying therapeutic intent, intervention objectives, or neuromodulation modality. Through these capabilities, sensors 136 provide additional non-intrusive inputs that support assessment of engagement and execution fidelity and contribute to longitudinal monitoring of adherence over repeated intervention sessions.

In some embodiments, the headset and system 126 may include a communication interface 138 configured to transmit data between components of the headset and system 126 and the Clarity Network 102. The communication interface 138 may enable transfer of data captured by the microphone 132, eye-tracking component 134, and sensors 136 to the Clarity Network 102 for processing, analysis, and storage. In some embodiments, the communication interface 138 may further transmit information indicative of execution of intervention protocols, including session timing, task progression, completion status, interruptions, and applied engagement-supporting adjustments, to support logging of session activity within the patient database 112 and longitudinal tracking of adherence and delivery fidelity over time. The communication interface 138 may support wired or wireless communication using one or more communication protocols and may operate continuously or intermittently during engaging neuromodulation sessions. In some embodiments, the communication interface 138 may facilitate real-time or near-real-time transmission of engagement-related data or execution-related information to support monitoring and adaptation of session delivery by the engagement module 140. In some embodiments, the communication interface 138 may further support transmission of control signals, configuration data, or content updates from the Clarity Network 102 to the headset and system 126, enabling coordinated operation of content delivery, sensing, and engagement-supporting functions during engaging neuromodulation sessions. Collectively, the components illustrated in FIG. 1 enable closed-loop monitoring and control of interactive neuromodulation-based intervention delivery, in which execution fidelity and longitudinal compliance are continuously assessed and supported through adaptive engagement mechanisms layered on top of intervention protocols.

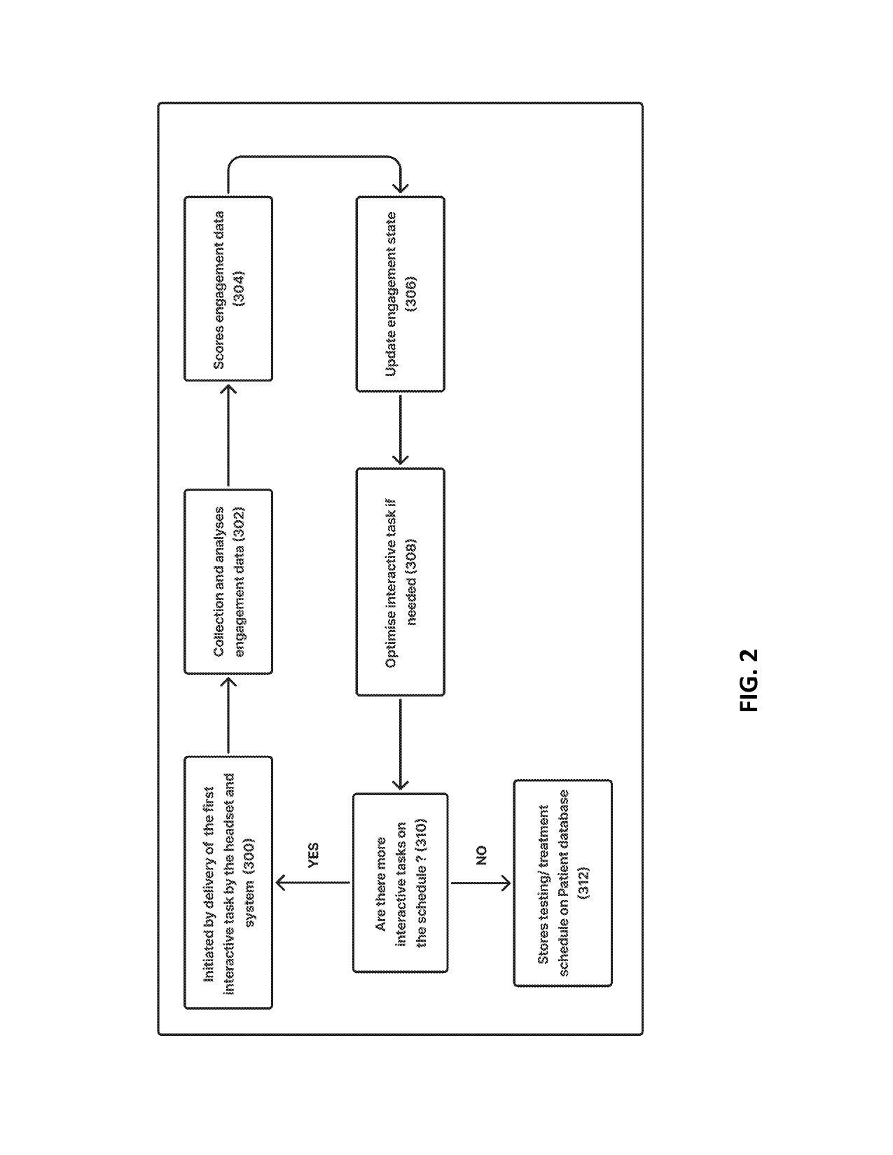

FIG. 2 illustrates an example method implemented by the treatment module 114 for configuring, delivering, monitoring, and updating an engaging neuromodulation session using patient-related information and engagement-related feedback. The method begins at step 200, initiated based on information retrieved from the patient database 112, which may include user identifiers, session history, configuration parameters, scheduling information, or other patient-specific information previously stored by the system. In some embodiments, initiation at step 200 may further include receiving one or more session selection inputs via the headset and system 126, including selection of a session type, scheduled session identifier, or user-facing preferences, and verifying readiness of one or more sensing components prior to session execution. For example, the system may verify availability or signal quality for one or more of the microphone 132, eye-tracking component 134, or sensors 136, and may prompt a brief calibration, fit adjustment, or confirmation step prior to transmission of the first task at step 208, thereby supporting reliable capture of engagement-related inputs during the session. At step 202, the treatment module 114 determines a schedule of therapeutic tasks or intervention segments to be delivered during an engaging neuromodulation session, including selection and sequencing of content elements retrieved from the VR database 116, task definitions and execution rules retrieved from the task logic database 118, and stimulation protocols retrieved from the neurostimulation database 122. At step 204, the treatment module 114 determines a gamification strategy by selecting engagement-supporting elements from the gamification database 120 and configuring such elements to operate as a layer applied to the scheduled tasks without altering the underlying task objectives or stimulation protocols defined for the session. At step 206, the treatment module 114 stores the resulting gamified therapeutic schedule in the memory 110, including association with patient-specific identifiers and session structure. At step 208, the system initiates delivery of the schedule by transmitting a first gamified therapeutic task to the headset and system 126 for execution as part of an engaging neuromodulation session. During execution, engagement-related data is captured and analyzed at step 210 based on inputs from one or more headset components, including the microphone 132, eye-tracking component 134, and sensors 136, and such data may be indicative of user interaction, attention, responsiveness, execution fidelity, or continuity of participation. Based on the engagement-related data, the engagement module 140 may determine whether engagement or execution criteria are satisfied and, if criteria are not satisfied, may initiate optimization at step 212 by updating engagement-supporting elements associated with the gamification strategy, including adjustment of feedback timing, prompts, pacing, reinforcement cues, or presentation characteristics, and in some embodiments adjustment of delivery-related presentation characteristics, while preserving the therapeutic intent and stimulation protocol defined for the session. At step 214, a determination is made whether additional gamified therapeutic tasks remain on the schedule; if additional tasks remain, the method returns to step 208 to transmit a next task to the headset and system 126. If no further tasks remain, the gamification strategy, engagement measures, and session metadata are stored at step 216 in the patient database 112 as part of a longitudinal record associated with the user. In this manner, FIG. 2 illustrates a delivery process in which an engaging neuromodulation session is delivered through the headset and system 126 while engagement, execution fidelity, and compliance are monitored and supported through adaptive engagement mechanisms layered on top of a predefined session structure.

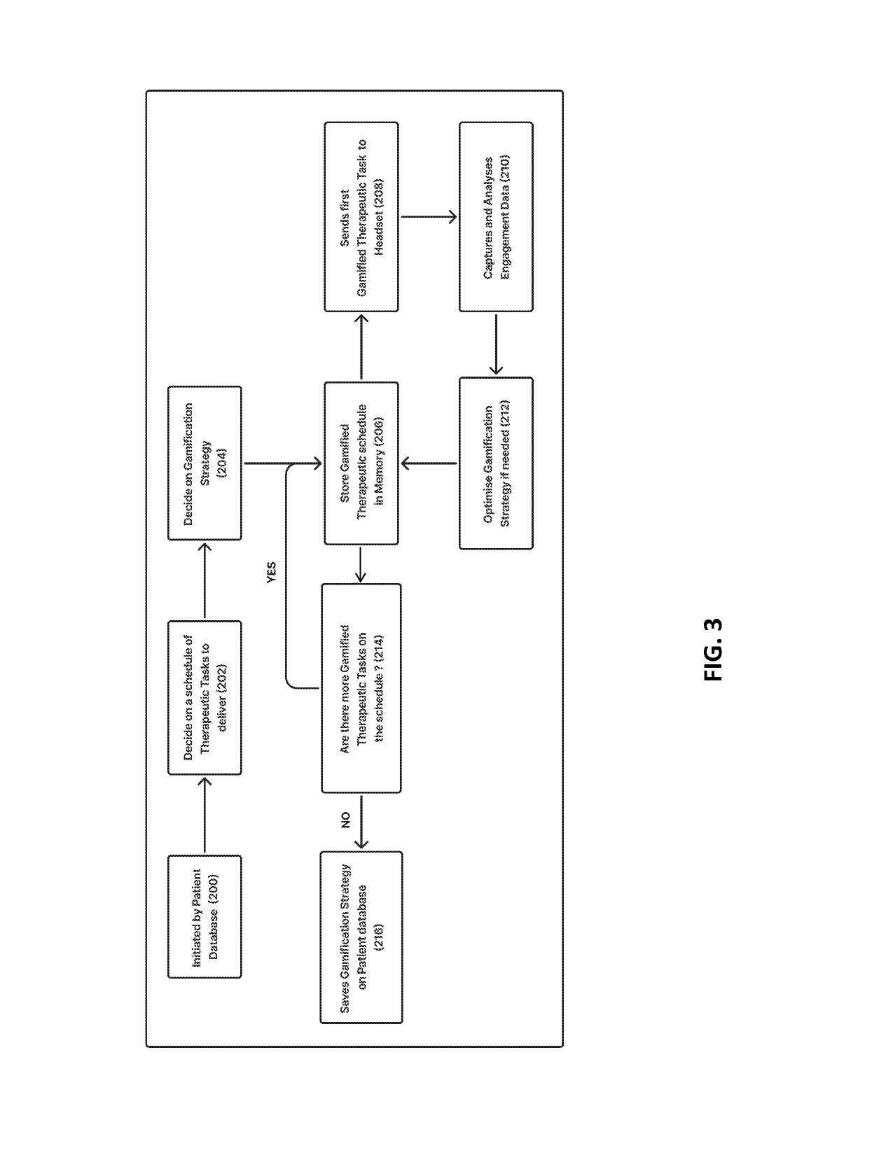

FIG. 3 illustrates an example operational flow of an engagement module 140 configured to monitor, score, and manage user engagement during engaging neuromodulation sessions in order to support session completion, execution fidelity, and longitudinal adherence. In some embodiments, operation of the engagement module 140 may be initiated upon delivery of the first task scheduled for an engaging neuromodulation session, at step 300, wherein the engaging neuromodulation session comprises one or more interactive therapeutic tasks delivered via the headset and system 126. During execution of a given interactive task, the engagement module 140 may receive engagement-related input data captured by one or more components of the headset and system 126, at step 302, wherein such input data may include, by way of example and not limitation, audio data captured by one or more microphones 132, gaze and eyelid data captured by an eye-tracking component 134, physiological or neural signals captured by sensors 136 such as pupillometry sensors or electrodes, and motion- or interaction-related data captured by additional sensors. The engagement module 140 may process the received engagement-related input data to extract one or more engagement-related features, at step 304, wherein such processing may include, by way of example and not limitation, signal processing, time-series analysis, feature extraction, normalization, aggregation, or other analytical operations, and may be performed using processing resources implemented locally, remotely, or in a distributed computing environment; extracted features may reflect indicators such as responsiveness to prompts, reaction times, continuity of interaction, gaze stability, frequency of interruptions, arousal-state proxies, or signs of reduced alertness, and based on one or more such extracted features the engagement module 140 may compute one or more engagement or compliance scores consistent with the scoring operation illustrated at step 304. By way of example and not limitation, a composite score for arousal or alertness may be computed based on a combination of eye-tracking metrics (e.g., fixation stability, blink rate, or prolonged eyelid closure), physiological measures such as heart-rate variability or pupillometry, neural indicators such as slow-wave activity or changes in spectral power derived from EEG signals, reaction-time distributions obtained from task performance, and speech-derived features such as response latency, voice cadence, or prosodic variability; in some embodiments, elevated stress or cognitive load scores may be inferred from concurrent increases in physiological arousal markers, such as but not limited to irregular gaze behavior, increased response variability, increased heart rate, or changes in vocal dynamics, relative to individualized baselines. In some embodiments, the computed engagement or compliance scores may represent composite measures derived from multiple input modalities and may be indicative of overall session engagement, attentional state, execution fidelity, or likelihood of session completion; engagement scores may be evaluated relative to predefined thresholds, protocol-defined criteria, historical baselines, or longitudinal engagement profiles stored in the patient database 112, and engagement scores stored in the patient database 112 may further be associated with confidence, validity, or execution-quality indicators reflecting the conditions under which session data was obtained, thereby supporting downstream interpretation of results. The engagement module 140 may update, at step 306, an engagement state record comprising intermediate features, rolling-window metrics, or session-context variables stored in working memory, buffers, or caches to support real-time or near-real-time control of prompts, feedback, and adaptations during the engaging neuromodulation session. At step 308, when engagement optimization is initiated, the engagement module 140 may cause one or more adaptations to be applied to the engaging neuromodulation session, wherein such adaptations may include, by way of example and not limitation, modification of engagement-supporting elements including elements retrieved from the gamification database 120, adjustment of feedback frequency or modality, introduction of prompts or reminders, alteration of pacing, or modification of virtual environment characteristics retrieved from the virtual reality database 116, and in some embodiments engagement optimization may further include adjustment of task presentation parameters or interaction flow defined by the task logic database 118, provided such adjustments remain consistent with the intended session objective and do not introduce new therapeutic logic during a session. In some embodiments, machine-learning or artificial-intelligence techniques may be used by the engagement module 140 to perform extraction, selection, or weighting of engagement-related features from multimodal input data, and additionally to determine, select, or optimize engagement-related adaptations applied during an engaging neuromodulation session. Following engagement optimization, the engagement module 140 may determine, at step 310, whether additional interactive tasks remain on the session schedule; if additional tasks remain, execution may proceed to delivery of the next scheduled interactive task and engagement monitoring may continue as described above, and if no additional tasks remain, the engagement module 140 may cause session-level engagement outputs, execution-fidelity indicators, and associated session metadata (including schedule completion information) to be stored in the patient database 112, at step 312. In some embodiments, the outcomes stored in the patient database 112 at step 312 may include (i) a primary outcome value associated with task execution and (ii) an accompanying fidelity or confidence value, such that downstream use of the outcome accounts for reduced attention, reduced arousal, inconsistent responsiveness, or other indicators of incomplete or low-quality task execution; in some embodiments, the system may apply a weighting, adjustment, flag, or penalty to the outcome based on engagement-related measures, may recommend repetition or rescheduling of one or more tasks, or may present the outcome as conditionally valid based on whether execution fidelity satisfies predefined criteria. By way of example and not limitation, a patient undergoing home-based neuromodulation therapy may participate in daily engaging neuromodulation sessions comprising audiovisual sensory stimulation embedded within a virtual environment that is paired with a task logic probing memory, and a potential output from the memory task may consist of a recall score and an average response latency; if the engagement module 140 detects repeated prolonged eye closure and extended gaze diversion during stimulus presentation, the system may store the recall score together with a reduced confidence indicator and/or flag the task as low-fidelity, thereby indicating that the measured performance may reflect reduced attention or arousal rather than capability alone. In some embodiments, analogous engagement, execution-fidelity, or confidence indicators may be generated for delivery of session content incorporating sensory stimulation or task-based therapeutic content, wherein such indicators reflect the degree to which prescribed session content was delivered under conditions consistent with intended execution. In some embodiments, engagement-related outputs generated by the engagement module 140 may be used to initiate adaptive responses at different levels of granularity during an engaging neuromodulation session, including task-level, session-level, or longitudinal engagement adaptations; at a task level, engagement-related measures may be used to adapt task difficulty or interaction demands via the task logic database 118, such as reducing difficulty, adjusting pacing, or modifying response timing requirements, in order to facilitate continued participation and completion while preserving the task identity and session objective; at a session level, engagement-related outputs may be used to modify presentation context or experiential elements to support motivation and sustained participation, including selecting or transitioning to virtual environments with different content attributes retrieved from the VR database 116 or adjusting audiovisual presentation characteristics, and in some embodiments such selection may be informed by models trained on prior session data; at a longitudinal engagement level, engagement-related measures may inform adaptation of engagement strategies across repeated sessions over time, including modifying gamification structures retrieved from the gamification database 120 such as reward schedules, streak mechanics, or progression framing to support adherence across repeated sessions over time, without altering the underlying neuromodulation modality or therapeutic intent of a given session. In some embodiments, one or more of the above adaptations may be determined using artificial intelligence or machine-learning techniques trained on engagement, performance, and adherence data collected across sessions. In this manner, the engagement module 140 enables sustained compliance and delivery fidelity in interactive neuromodulation-based interventions by adapting engagement-supporting elements in response to observed user interaction, while maintaining separation between engagement optimization and therapeutic protocol definition.

What is claimed is:

1. A method of enhancing user compliance in virtual reality (VR) stimulation therapy, the method comprising:
storing information in memory regarding one or more modifications to interactive content, the modifications associated with different types of neurostimulation;
monitoring one or more interactive tasks performed in real-time during an interactive VR session established with a user device, wherein monitoring the interactive tasks includes measuring one or more performance parameters of a user performing the interactive tasks;
identifying that the performance parameters measured during the performance of the interactive tasks are indicative of a need for an identified type of neurostimulation; and
adjusting VR content in accordance with the modification associated with the identified type of neurostimulation, wherein the adjusted VR content is incorporated into a VR environment presented at a VR headset associated with the user device during the interactive VR session.

2. The method of claim 1, wherein the content element includes a gamification content element associated with the identified type of neurostimulation.

3. The method of claim 1, wherein the content associated with at least one virtual reality environment is tailored to an associated user.

4. The method of claim 3, further comprising generating the tailored content based on stored data specific to the associated user.

5. The method of claim 1, wherein the performance parameters include physiological parameters of the user measured by one or more sensors during performance of the interactive tasks.

6. The method of claim 1, further comprising storing data specific to a user, the data including one or more of historical VR session data, historical task performance data, historical scores, associated neurostimulation, and wherein identifying the need for the identified type of neurostimulation is further based on the stored data specific to the user.

7. The method of claim 1, wherein the VR content is adjusted dynamically in real-time during the VR session as the need for the identified type of neurostimulation is identified.

8. The method of claim 1, wherein adjusting the VR content further includes incorporating content associated with memory-based images, videos, narratives, music, sounds, locations, and scenes.

9. The method of claim 1, further comprising training a learning model to correlate the performance parameters of the user to one or more of the modifications corresponding to the different types of neurostimulation.

10. The method of claim 9, further comprising storing information regarding a therapeutic goal associated with a trend in the performance parameters, wherein identifying the need for the identified type of neurostimulation is further based on the therapeutic goal.

11. A system of enhancing user compliance in virtual reality (VR) stimulation therapy, the system comprising:
memory that stores information regarding one or more modifications to interactive content, the modifications associated with different types of neurostimulation; and
a communication interface that communicates with a user device over a communication network, wherein the communication interface receives monitoring data regarding one or more interactive tasks performed in real-time during an interactive VR session established with the user device, wherein the monitoring data regarding the interactive tasks includes measurements of one or more performance parameters of a user performing the interactive tasks; and
a processor that executes instructions stored in memory, wherein the processor executes the instructions to:
identify that the performance parameters measured during the performance of the interactive tasks are indicative of a need for an identified type of neurostimulation, and
adjust VR content in accordance with the modification associated with the identified type of neurostimulation, wherein the adjusted VR content is incorporated into a VR environment presented at a VR headset associated with the user device during the interactive VR session.

12. The system of claim 11, wherein the content element includes a gamification content element associated with the identified type of neurostimulation.

13. The system of claim 11, wherein the content associated with at least one virtual reality environment is tailored to an associated user.

14. The system of claim 13, wherein the processor executes further instructions to generate the tailored content based on stored data specific to the associated user.

15. The system of claim 11, wherein the performance parameters include physiological parameters of the user measured by one or more sensors during performance of the interactive tasks.

16. The system of claim 11, wherein the memory further stores data specific to a user, the data including one or more of historical VR session data, historical task performance data, historical scores, associated neurostimulation, and wherein identifying the need for the identified type of neurostimulation is further based on the stored data specific to the user.

17. The system of claim 11, wherein the processor adjusts the VR content dynamically in real-time during the VR session as the need for the identified type of neurostimulation is identified.

18. The system of claim 11, wherein the processor adjusts the VR content by incorporating content associated with memory-based images, videos, narratives, music, sounds, locations, and scenes.

19. The system of claim 11, wherein the processor executes further instructions to train a learning model to correlate the performance parameters of the user to one or more of the modifications corresponding to the different types of neurostimulation.

20. The system of claim 19, wherein the memory further stores information regarding a therapeutic goal associated with a trend in the performance parameters, and wherein the processor identifies the need for the identified type of neurostimulation further based on the therapeutic goal.
